(19) United States
(12) Patent Application Publication
(10) Pub. No.: US 2020/0338014 A1
(43) Pub. Date: Oct. 29, 2020

(54) MONOTERPENE COMPOSITIONS AND USES THEREOF

(71) Applicant: C & J Lab Pty Ltd., Chatswood, NSW (AU)

(72) Inventor: Qi Wang, Annerley, Queensland (AU)

(21) Appl. No.: 16/643,006

(22) PCT Filed: Aug. 28, 2017
(86) PCT No.: PCT/US2017/048943

§ 371 Date: Feb. 28, 2020

(51) Int. Cl. A61K 31/015; A61K 31/045; A61K 31/352

(52) U.S. Cl. CPC A61K 31/015; A61K 31/352; A61K 31/045

(57) ABSTRACT

Provided herein are compositions and pharmaceutical compositions comprising α-terpinolene, terpinen-4-ol, and α-terpineol. Also provided herein are methods of using such compositions.

### BACKGROUND

Antibiotic resistance is rising to high levels in all parts of the world. New resistance mechanisms are emerging and spreading globally, threatening abilities to treat common infectious diseases. A growing number of infections are becoming harder, and sometimes impossible, to treat as antibiotics become less effective. Where antibiotics can be bought for human or animal use without a prescription, emergence and spread of resistance is made worse. Similarly, in countries without standard treatment guidelines, antibiotics are often over-prescribed by health workers and veterinarians and over-used by the public.

Antibiotic resistance is accelerated by misuse and overuse of antibiotics, as well as poor infection prevention and control. Without alternative therapies, a post-antibiotic era, in which common infections and minor injuries can once again kill, is increasingly possible. Accordingly, there exists a need for alternative therapies to antibiotics.

### SUMMARY

Provided herein are compositions comprising monoterpenes useful in treating a disease (e.g., bacterial disease or infection) in a livestock.

In some aspects, provided herein are compositions comprising α-terpinolene, terpinen-4-ol, and α-terpineol.

In some aspects, provided herein are methods of decreasing the rate of mortality in a subject in need thereof, comprising administering a composition provided herein to the subject.

In some aspects, provided herein are methods of decreasing the rate of bacterial infection in a subject in need thereof, comprising administering a composition provided herein to the subject.

In some aspects, provided herein are methods of decreasing the rate of diarrhea in a subject in need thereof, comprising administering a composition provided herein to the subject.

In some aspects, provided herein are methods of decreasing the rate of premature death in a subject in need thereof, comprising administering a composition provided herein to the subject.

In some aspects, provided herein are methods of treating diarrhea in a subject in need thereof, comprising administering a composition provided herein to the subject.

### DETAILED DESCRIPTION

Provided herein are compositions useful as alternatives to antibiotic agents. In some embodiments, the compositions provided herein are useful in the livestock industry. In some embodiments, administration of the compositions provided herein to a subject (e.g., a livestock) improve feed to subject mass conversion ratios, lead to healthier livestock, reduce diarrhea in livestock (e.g., in porcine clades), reduce rates of livestock mortality, increase livestock production efficiency, or enhance immune system functionality. In some embodiments, the compositions provided herein, can be organic, are cost-effective, and typically stable under standard temperature and pressure conditions.

Melaleuca alternifolia extract, commonly known as tea tree oil (TTO), has been associated with therapeutic properties. However, the toxicity of TTO restricts its usage to topical administration. The compositions provided herein include monoterpenes that are found in TTO. However, in contrast to the toxicity of TTO, the compositions provided herein are suitable for not only topical administration, but systemic administration as well. It has surprisingly been found that, the compositions provided herein have an LD₅₀ of 5,000 mg/kg or higher.

### Definitions

Listed below are definitions of various terms used to describe the compositions and methods provided herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which the compositions and methods provided herein pertain. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include”, “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed compositions and methods.

As used herein, the terms “effective amount,” “pharmaceutically effective amount,” and “therapeutically effective amount” refer to a nontoxic but sufficient amount of an agent to provide the desired biological result. That result may be reduction, alleviation, or both, of the signs, symptoms, or causes of a disease, or any other desired alteration of a biological system. An appropriate therapeutic amount in any individual case may be determined by one of ordinary skill in the art using routine experimentation.

As used herein, the term “extreme difference” refers to a statistical analysis wherein a calculated p-value, sometimes referred to as a probability value, is less than 0.01 (e.g., p<0.01).

As used herein, the term “feed conversion efficiency” refers to a ratio or rate measuring the efficiency with which the bodies of livestock convert animal feed into the desired output. For dairy cows, for example, the output is milk, whereas in animals raised for meat (such as beef cows, pigs, chickens, and fish) the output is the flesh, that is, the body mass gained by the animal, represented either in the final mass of the animal or the mass of the dressed output. FCE is the mass of the output divided by the input (thus mass of milk or meat per mass of feed).

As used herein, the terms “feed conversion ratio,” “feed conversion rate,” or “feed to subject mass conversion ratio” refer to a ratio or rate measuring the efficiency with which the bodies of livestock convert animal feed into the desired output. For dairy cows, for example, the output is milk, whereas in animals raised for meat (such as beef cows, pigs, chickens, and fish) the output is the flesh, that is, the body mass gained by the animal, represented either in the final mass of the animal or the mass of the dressed output. FCR is the mass of the input divided by the output (thus mass of feed per mass of milk or meat).

As used herein, the term “meat tenderness” refers to meat resistance to shear force. The resistance of meat to shear force can be gauged by, for example, a Slice Shear Force test or a Warner-Bratzler Shear Force test.

As used herein, the terms “% area by gas chromatography” or “A_{GC}%” refers to the integrated area, as a percentage, of one or more compounds as determined by gas chromatography using a flame ionization detector in accordance with ISO 4730:2004.

As used herein, the term “premature death” refers to death that occurs before a subject reaches an expected age (e.g., maturity).

As used herein, the term “pharmaceutically acceptable” refers to a material, such as an excipient or diluent, which does not abrogate the biological activity or properties of the compound or composition, and is relatively non-toxic, i.e., the material may be administered to a subject without causing undesirable biological effects or interacting in a deleterious manner with any of the components of the composition in which it is contained.

As used herein, the term “pharmaceutically acceptable excipient” means a pharmaceutically acceptable material of a functional category including acidifying agent, aerosol propellant, air displacement, alcohol denaturant, alkalizing agent, anticaking agent, antifoaming agent, antimicrobial preservative, antioxidant, buffering agent, bulking agent, capsule lubricant, chelating agent, coating agent, coloring agent, complexing agent, desiccant, emollient, emulsifying agent, solubilizing agent, filtering aid, flavoring agent, perfume agent, glidant, humectant, ointment base, plasticizer, polymer membrane, sequestering agent, solvent, sorbent, stiffening agent, suppository base, suspending agent, viscosity-increasing agent, sweetening agent, binder, diluent, disintegrant, lubricant, tonicity agent, vehicle, water repelling agent, or wetting agent involved in carrying or transporting a composition provided herein within or to the subject such that the composition may perform its intended function. Typically, such constructs are carried or transported from one organ, or portion of the body, to another organ, or portion of the body. Each excipient must be “acceptable” in the sense of being compatible with the other ingredients of the formulation, including the components of the compositions provided herein, and not injurious to the subject. As used herein, “pharmaceutically acceptable excipient” also includes any and all coatings, antibacterial and antifungal agents, and absorption delaying agents, and the like that are compatible with the activity of the compositions provided herein, and are physiologically acceptable to the subject. Supplementary active compounds may also be incorporated into the compositions. Pharmaceutically acceptable excipients are known in the art and described, for example in Remington's Pharmaceutical Sciences (Genaro, Ed., Mack Publishing Co., 1985, Easton, Pa.) and The United States Pharmacopeia: The National Formulary (The United States Pharmacopeial Convention, 2009, Rockville, Md.), each of which are incorporated herein by reference.

As used herein, the term “pharmaceutical composition” refers to a composition provided herein with a pharmaceutically acceptable excipient. The pharmaceutical composition facilitates administration of the composition to a subject. Multiple techniques of administering a composition exist in the art, which include, in some embodiments, intravenous, oral, aerosol, parenteral, ophthalmic, pulmonary, and topical administration.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “significant difference” refers to a statistical analysis wherein a calculated p-value, sometimes referred to as a probability value, is less than 0.05 (e.g., p<0.05).

As used herein, the term “subject” refers to a human or a non-human subject. Non-human subjects include, in some embodiments, livestock and pets, such as ovine, bovine, porcine, canine, feline and murine mammals.

As used herein, the term “treatment” or “treating,” is defined as the application or administration of a therapeutic agent, i.e., a composition provided herein (alone or in combination with another pharmaceutical agent), to a subject, or application or administration of a therapeutic agent to an isolated tissue or cell line from a subject (e.g., for diagnosis or ex vivo applications), who has a disease or infection, with the purpose to cure, heal, alleviate, relieve, alter, remedy, ameliorate, improve or affect the disease or infection, the symptoms of the disease or infection, or the potential to develop the disease or infection. Such treatments may be specifically tailored or modified, based on knowledge obtained from the field of pharmacogenomics.

### Compositions

Antibiotic resistance poses a major threat to the use of current antibiotic therapies (e.g., for livestock production). Cross-class combination therapy is one strategy for delaying emergence of drug resistant bacterial strains. The compositions provided herein, for example, can, when administered alone or in combination with other antibacterial therapies, offer a reduction of antibiotic resistance profiles and improved management of livestock health.

In one aspect, provided herein is a composition comprising: α-terpinolene; terpinen-4-ol; and α-terpineol; wherein the molar ratio of α-terpinolene:terpinen-4-ol:α-terpineol is about 1:(about 2.15 to about 3.65):(about 0.22 to about 0.65).

In some embodiments, the molar ratio of α-terpinolene:terpinen-4-ol:α-terpineol is about 1:(about 2.35 to about 3.25):(about 0.27 to about 0.52). In some embodiments, the molar ratio of α-terpinolene:terpinen-4-ol:α-terpineol is about 1:(about 2.5 to about 2.85):(about 0.32 to about 0.45).

In some embodiments, the composition comprises about 17 to about 25 A_{GC}% α-terpinolene. In some embodiments, the composition comprises about 17, 18, 18.6, 19, 19.6, 20, 20.6, 21, 22, 23, 24, 25, 17 to 19, 18 to 20, 19 to 21, 20 to 22, 21 to 23, 22 to 24, 23 to 25, 17 to 20, 19 to 23, 18 to 21, 18.6 to 19.6, 19.6 to 20.6, 18 to 19.6, 19.6 to 21, or 20 to 25 A_{GC}% A_{GC}% α-terpinolene, or any range bounded by any two of these values. In some embodiments, the composition comprises about 19.6 A_{GC}% α-terpinolene.

In some embodiments, the composition comprises about 45 to about 58 A_{GC}% terpinen-4-ol. In some embodiments, the composition comprises about 45, 46, 47, 48, 49, 50, 51, 51.5, 52, 52.5, 53, 53.5, 54, 55, 56, 57, 58, 45 to 47, 46 to 48, 47 to 49, 48 to 50, 49 to 51, 50 to 52, 51 to 53, 52 to 54, 53 to 55, 54 to 56, 55 to 57, 56 to 58, 45 to 50, 50 to 54, 54 to 58, 51 to 53, 50 to 52.5, 51 to 52.5, 51.5 to 52.5, 52.5 to 53.5, 52.5 to 54, 52.5 to 55, 51 to 55, or 55 to 58 A_{GC}% terpinen-4-ol, or any range bounded by any two of these values. In some embodiments, the composition comprises about 52.5 A_{GC}% terpinen-4-ol.

In some embodiments, the composition comprises about 2.0 to about 11 A_{GC}% α-terpineol. In some embodiments, the composition comprises about 2, 3, 4, 5, 6, 7, 8, 9, 10, 11, 2 to 4, 3 to 5, 4 to 6, 5 to 7, 6 to 8, 7 to 9, 8 to 10, 9 to 11, 6 to 11, 8 to 11, 8.0 to 9.2, 8.2 to 9.2, 9.2 to 10.2, 9.2 to 11.0, or 9.0 to 10.0 A_{GC}% α-terpineol, or any range bounded by any two of these values. In some embodiments, the composition comprises about 9.2 A_{GC}% α-terpineol.

In some embodiments, the composition comprises about 0.5 to about 2.5 A_{GC}% 1,8-cineole. In some embodiments, the composition comprises about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 0.5 to 1.0, 1.0 to 1.5, 1.5 to 2.0, 2.0 to 2.5, 1.0 to 1.8, 1.5 to 1.8, 1.8 to 2.0, 1.8 to 2.5, or 1.4 to 2.1 A_{GC}% 1,8-cineole, or any range bounded by any two of these values. In some embodiments, the composition comprises about 1.8 A_{GC}% 1,8-cineole.

In some embodiments, the composition comprises about 0.5 to about 2.5 A_{GC}% γ-terpinene. In some embodiments, the composition comprises about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 0.5 to 1.0, 1.0 to 1.5, 1.5 to 2.0, 2.0 to 2.5, 1.5 to 2.2, 2.0 to 2.2, 2.2 to 2.5, 1.8 to 2.5, or 1.5 to 2.5 A_{GC}% γ-terpinene, or any range bounded by any two of these values. In some embodiments, the composition comprises about 2.2 A_{GC}% γ-terpinene.

In some embodiments, the composition comprises α-pinene up to about 1.7 A_{GC}%. In some embodiments, the composition comprises about 0.001, 0.01, 0.1, 0.2, 0.3, 0.4, 0.5, 0.6, 0.7, 0.8, 0.9, 1.0, 1.1, 1.2, 1.3, 1.4, 1.5, 1.6, 1.7, 0.1 to 0.5, 0.5 to 0.9, 0.9 to 1.3, 1.3 to 1.7, 0.5 to 1.7, 0.5 to 1.6, 1.0 to 1.6, 1.3 to 1.6, 1.4 to 1.7, 1.6 to 1.7, 0.001 to 1.7, 0.01 to 1.7, 0.1 to 1.7, or 1.0 to 1.7 A_{GC}% α-pinene, or any range bounded by any two of these values. In some embodiments, the composition comprises about 1.6 A_{GC}% α-pinene.

In some embodiments, the composition comprises sabinene up to about 0.2 A_{GC}%. In some embodiments, the composition comprises about 0.001, 0.01, 0.1, 0.2, 0.001 to 0.01, 0.001 to 0.1, 0.001 to 0.2, 0.01 to 0.1, 0.01 to 0.2, or 0.1 to 0.2 A_{GC}% sabinene, or any range bounded by any two of these values. In some embodiments, the composition comprises about 0.001 A_{GC}% sabinene. In some embodiments, the composition comprises about 0.01 A_{GC}% sabinene. In some embodiments, the composition comprises about 0.1 A_{GC}% sabinene. In some embodiments, the composition comprises sabinene up to about 0.001 A_{GC}%. In some embodiments, the composition comprises sabinene up to about 0.01 A_{GC}%.

In some embodiments, the composition comprises α-terpinene up to about 0.2 A_{GC}%. In some embodiments, the composition comprises about 0.001, 0.01, 0.1, 0.2, 0.001 to 0.01, 0.001 to 0.1, 0.001 to 0.2, 0.01 to 0.1, 0.01 to 0.2, or 0.1 to 0.2 A_{GC}% α-terpinene, or any range bounded by any two of these values. In some embodiments, the composition comprises about 0.001 A_{GC}% α-terpinene. In some embodiments, the composition comprises about 0.01 A_{GC}% α-terpinene. In some embodiments, the composition comprises about 0.1 A_{GC}% α-terpinene. In some embodiments, the composition comprises α-terpinene up to about 0.001 A_{GC}%. In some embodiments, the composition comprises α-terpinene up to about 0.01 A_{GC}%.

In some embodiments, the composition comprises limonene up to about 0.2 A_{GC}%. In some embodiments, the composition comprises about 0.001, 0.01, 0.1, 0.2, 0.001 to 0.01, 0.001 to 0.1, 0.001 to 0.2, 0.01 to 0.1, 0.01 to 0.2, or 0.1 to 0.2 A_{GC}% limonene, or any range bounded by any two of these values. In some embodiments, the composition comprises about 0.001 A_{GC}% limonene. In some embodiments, the composition comprises about 0.01 A_{GC}% limonene. In some embodiments, the composition comprises about 0.1 A_{GC}% limonene. In some embodiments, the composition comprises limonene up to about 0.001 A_{GC}%. In some embodiments, the composition comprises limonene up to about 0.01 A_{GC}%.

In some embodiments, the composition comprises p-cymene up to about 0.2 A_{GC}%. In some embodiments, the composition comprises about 0.001, 0.01, 0.1, 0.2, 0.001 to 0.01, 0.001 to 0.1, 0.001 to 0.2, 0.01 to 0.1, 0.01 to 0.2, or 0.1 to 0.2 A_{GC}% p-cymene, or any range bounded by any two of these values. In some embodiments, the composition comprises about 0.001 A_{GC}% p-cymene. In some embodiments, the composition comprises about 0.01 A_{GC}% p-cymene. In some embodiments, the composition comprises about 0.1 A_{GC}% p-cymene. In some embodiments, the composition comprises p-cymene up to about 0.001 A_{GC}%. In some embodiments, the composition comprises p-cymene up to about 0.01 A_{GC}%.

In some embodiments, the composition comprises aromadendren up to about 0.5 A_{GC}%. In some embodiments, the composition comprises about 0.001, 0.01, 0.1, 0.2, 0.5, 0.001 to 0.01, 0.001 to 0.1, 0.001 to 0.2, 0.001 to 0.5, 0.01 to 0.1, 0.01 to 0.2, 0.01 to 0.5, 0.1 to 0.2, 0.1 to 0.5, or 0.2 to 0.5 A_{GC}% aromadendren, or any range bounded by any two of these values. In some embodiments, the composition comprises about 0.001 A_{GC}% aromadendren. In some embodiments, the composition comprises about 0.01 A_{GC}% aromadendren. In some embodiments, the composition comprises about 0.1 A_{GC}% aromadendren. In some embodiments, the composition comprises aromadendren up to about 0.001 A_{GC}%. In some embodiments, the composition comprises aromadendren up to about 0.01 A_{GC}%.

In some embodiments, the composition comprises ledene up to about 0.5 A_{GC}%. In some embodiments, the composition comprises about 0.001, 0.01, 0.1, 0.2, 0.5, 0.001 to 0.01, 0.001 to 0.1, 0.001 to 0.2, 0.001 to 0.5, 0.01 to 0.1, 0.01 to 0.2, 0.01 to 0.5, 0.1 to 0.2, 0.1 to 0.5, or 0.2 to 0.5 A_{GC}% ledene, or any range bounded by any two of these values. In some embodiments, the composition comprises about 0.001 A_{GC}% ledene. In some embodiments, the composition comprises about 0.01 A_{GC}% ledene. In some embodiments, the composition comprises about 0.1 A_{GC}% ledene. In some embodiments, the composition comprises ledene up to about 0.001 A_{GC}%. In some embodiments, the composition comprises ledene up to about 0.01 A_{GC}%.

In some embodiments, the composition comprises δ-cadinene up to about 0.5 A_{GC}%. In some embodiments, the composition comprises about 0.001, 0.01, 0.1, 0.2, 0.5, 0.001 to 0.01, 0.001 to 0.1, 0.001 to 0.2, 0.001 to 0.5, 0.01 to 0.1, 0.01 to 0.2, 0.01 to 0.5, 0.1 to 0.2, 0.1 to 0.5, or 0.2 to 0.5 A_{GC}% δ-cadinene, or any range bounded by any two of these values. In some embodiments, the composition comprises about 0.001 A_{GC}% δ-cadinene. In some embodiments, the composition comprises about 0.01 A_{GC}% δ-cadinene. In some embodiments, the composition comprises about 0.1 A_{GC}% δ-cadinene. In some embodiments, the composition comprises δ-cadinene up to about 0.001 A_{GC}%. In some embodiments, the composition comprises δ-cadinene up to about 0.01 A_{GC}%.

In some embodiments, the composition comprises globulol up to about 0.5 A_{GC}%. In some embodiments, the composition comprises about 0.001, 0.01, 0.1, 0.2, 0.5, 0.001 to 0.01, 0.001 to 0.1, 0.001 to 0.2, 0.001 to 0.5, 0.01 to 0.1, 0.01 to 0.2, 0.01 to 0.5, 0.1 to 0.2, 0.1 to 0.5, or 0.2 to 0.5 A_{GC}% globulol, or any range bounded by any two of these values. In some embodiments, the composition comprises about 0.001 A_{GC}% globulol. In some embodiments, the composition comprises about 0.01 A_{GC}% globulol. In some embodiments, the composition comprises about 0.1 A_{GC}% globulol. In some embodiments, the composition comprises globulol up to about 0.001 A_{GC}%. In some embodiments, the composition comprises globulol up to about 0.01 A_{GC}%.

In some embodiments, the composition comprises virdiflorol up to about 0.5 A_{GC}%. In some embodiments, the composition comprises about 0.001, 0.01, 0.1, 0.2, 0.5, 0.001 to 0.01, 0.001 to 0.1, 0.001 to 0.2, 0.001 to 0.5, 0.01 to 0.1, 0.01 to 0.2, 0.01 to 0.5, 0.1 to 0.2, 0.1 to 0.5, or 0.2 to 0.5 A_{GC}% virdiflorol, or any range bounded by any two of these values. In some embodiments, the composition comprises about 0.001 A_{GC}% virdiflorol. In some embodiments, the composition comprises about 0.01 A_{GC}% virdiflorol. In some embodiments, the composition comprises about 0.1 A_{GC}% virdiflorol. In some embodiments, the composition comprises virdiflorol up to about 0.001 A_{GC}%. In some embodiments, the composition comprises virdiflorol up to about 0.01 A_{GC}%.

In some embodiments, the composition comprises about 17 to about 25 A_{GC}% α-terpinolene, about 45 to about 58 A_{GC}% terpinen-4-ol, about 2.0 to about 11 A_{GC}% α-terpineol, about 0.5 to about 2.5 A_{GC}% 1,8-cineole, or about 0.5 to about 2.5 A_{GC}% γ-terpinene.

In some embodiments, the composition comprises about 17 to about 25 A_{GC}% α-terpinolene, about 45 to about 58 A_{GC}% terpinen-4-ol, and about 2.0 to about 11 A_{GC}% α-terpineol.

In some embodiments, the composition comprises about 17 to about 25 A_{GC}% α-terpinolene, about 45 to about 58 A_{GC}% terpinen-4-ol, about 2.0 to about 11 A_{GC}% α-terpineol, about 0.5 to about 2.5 A_{GC}% 1,8-cineole, and about 0.5 to about 2.5 A_{GC}% γ-terpinene.

In some embodiments, the composition further comprises at least one of: α-pinene up to about 1.7 A_{GC}%; sabinene up to about 0.2 A_{GC}%; α-terpinene up to about 0.2 A_{GC}%; limonene up to about 0.2 A_{GC}%; p-cymene up to about 0.2 A_{GC}%; aromadendrene up to about 0.5 A_{GC}%; ledene up to about 0.5 A_{GC}%; δ-cadinene up to about 0.5 A_{GC}%; globulol up to about 0.5 A_{GC}%; or virdiflorol up to about 0.5 A_{GC}%.

In some embodiments, the composition comprises: about 17 to about 25 A_{GC}% α-terpinolene; about 45 to about 58 A_{GC}% terpinen-4-ol; about 2.0 to about 11 A_{GC}% α-terpineol; about 0.5 to about 2.5 A_{GC}% 1,8-cineole; about 0.5 to about 2.5 A_{GC}% γ-terpinene; α-pinene up to about 1.7 A_{GC}%; sabinene up to about 0.2 A_{GC}%; α-terpinene up to about 0.2 A_{GC}%; limonene up to about 0.2 A_{GC}%; p-cymene up to about 0.2 A_{GC}%; aromadendrene up to about 0.5 A_{GC}%; ledene up to about 0.5 A_{GC}%; δ-cadinene up to about 0.5 A_{GC}%; globulol up to about 0.5 A_{GC}%; and virdiflorol up to about 0.5 A_{GC}%.

In some embodiments, the α-terpinolene and terpinen-4-ol together are at least about 62 A_{GC}% of the composition.

In some embodiments, the α-terpinolene and terpinen-4-ol together are at least about 72 A_{GC}% of the composition.

In some embodiments, the α-terpinolene and terpinen-4-ol together are about 72 to about 83 A_{GC}% of the composition.

In some embodiments, the α-terpinolene and terpinen-4-ol together are about 62 to about 83 A_{GC}% of the composition.

In some embodiments, the α-terpinolene and terpinen-4-ol together are about 70 to about 75 A_{GC}% of the composition.

In some embodiments, the α-terpinolene, terpinen-4-ol, and α-terpineol together are at least about 62.5 A_{GC}% of the composition.

In some embodiments, the α-terpinolene, terpinen-4-ol, and α-terpineol together are at least about 78 A_{GC}% of the composition.

In some embodiments, the α-terpinolene, terpinen-4-ol, and α-terpineol together are about 78 to about 85.5 A_{GC}% of the composition.

In some embodiments, the α-terpinolene, terpinen-4-ol, and α-terpineol together are about 62.5 to about 85.5 A_{GC}% of the composition.

In some embodiments, the α-terpinolene, terpinen-4-ol, and α-terpineol together are about 75.5 to about 81 A_{GC}% of the composition.

In some embodiments, the composition comprises: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; and α-pinene about 1.6 A_{GC}%.

In some embodiments, the composition comprises: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some embodiments, the composition is a pharmaceutical composition.

In some embodiments, the composition further comprises at least one pharmaceutically acceptable excipient.

In some embodiments, provided herein is a solid mixture comprising: a composition provided herein, and an excipient (e.g., a pharmaceutically acceptable excipient).

In some embodiments, the pharmaceutically acceptable excipient is microcrystalline cellulose. In some embodiments, the ratio of the composition to the excipient is about 4:1 by weight. In some embodiments, the ratio of the composition to the excipient is about 6:1, 5:1, 4:1, 3:1, 1:1, 6:1 to 1:1, 5:1 to 3:1, 5:1 to 4:1, or 4:1 to 3:1 by weight.

In some embodiments, the composition comprises Formula I (Table 1) and microcrystalline cellulose. In some embodiments, the composition comprises Formula I and d-tocopherol polyethylene glycol 1000 succinate (TPGS-1000). In some embodiments, the composition comprises Formula I, d-tocopherol polyethylene glycol 1000 succinate (TPGS-1000), and water.

TABLE 1
composition of Formula I.
Formula I

| Component | A_{GC} % |
|---|---|
| α-Pinene | 1.60 |
| Sabinene | trace |
| α-Terpinene | trace |
| Limonene | trace |
| p-Cymene | trace |
| 1,8-Cineole | 1.80 |
| γ-Terpinene | 2.20 |
| Terpinolene | 19.6 |
| Terpinen-4-ol | 52.5 |
| α-Terpineol | 9.2 |
| Aromadendrene | trace |
| Ledene | trace |
| δ-Cadinene | trace |
| Globulol | trace |
| Viridiflorol | trace |

In some embodiments, provided herein is a liquid mixture comprising: a composition provided herein, and an excipient (e.g. a pharmaceutically acceptable excipient).

In some embodiments, the pharmaceutically acceptable excipient is d-tocopherol polyethylene glycol 1000 succinate. In some embodiments, the liquid mixture comprises about 4% by weight d-tocopherol polyethylene glycol 1000 succinate. In some embodiments, the liquid mixture comprises about 1% to about 10% (e.g., about 2% to about 6%) d-tocopherol polyethylene glycol 1000 succinate by weight. In some embodiments, the liquid mixture comprises about 2% to about 4% composition by weight. In some embodiments, the liquid mixture comprises about 0.5% to about 10% (e.g., about 1% to about 2%, about 2% to about 5%, or about 1% to about 5%) composition by weight. In some embodiments, the liquid mixture comprises about 1% to about 99.5% (e.g., about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 99.5%) water by weight. In some embodiments, the liquid mixture comprises about 96% to about 98% water by weight. In some embodiments, the liquid mixture comprises about 92% to about 98% water by weight.

### Methods

In some aspects, provided herein are methods of improving the feed to subject mass conversion ratio (feed conversion efficiency) in a subject in need thereof, comprising administering a composition provided herein to the subject.

In some aspects, provided herein are methods of improving weight gain in a subject in need thereof, comprising administering a composition provided herein to the subject.

In some aspects, provided herein are methods of improving myocardial function in a subject in need thereof, comprising administering a composition provided herein to the subject.

In some aspects, provided herein are methods of decreasing the rate of mortality in a subject in need thereof, comprising administering a composition provided herein to the subject.

In some aspects, provided herein are methods of decreasing the rate of bacterial infection in a subject in need thereof, comprising administering a composition provided herein to the subject.

In some aspects, provided herein are methods of decreasing the rate of diarrhea in a subject in need thereof, comprising administering a composition provided herein to the subject.

In some aspects, provided herein are methods of decreasing the rate of premature death in a subject in need thereof, comprising administering a composition provided herein to the subject.

In some aspects, provided herein are methods of treating a bacterial infection in a subject in need thereof, comprising administering a composition provided herein to the subject.

In some aspects, provided herein are methods of preventing a bacterial infection in a subject in need thereof, comprising administering a composition provided herein to the subject.

In some aspects, provided herein are methods of treating diarrhea in a subject in need thereof, comprising administering a composition provided herein to the subject.

In some aspects, provided herein are methods of treating colibacillosis in a subject in need thereof, comprising administering a composition provided herein to the subject.

In some aspects, provided herein are methods of treating coccidiosis in a subject in need thereof, comprising administering a composition provided herein to the subject.

In some aspects, provided herein are methods of treating swine dysentery in a subject in need thereof, comprising administering a composition provided herein to the subject.

In some aspects, provided herein are methods of preventing diarrhea in a subject in need thereof, comprising administering a composition provided herein to the subject.

In some aspects, provided herein are methods of treating liver disease in a subject in need thereof, comprising administering a composition provided herein to the subject.

In some aspects, provided herein are methods of treating immune disease in a subject in need thereof, comprising administering a composition provided herein to the subject.

In some aspects, provided herein are methods of improving the immune system in a subject in need thereof, comprising administering a composition provided herein to the subject.

In some aspects, provided herein are methods of increasing daily weight gain in a subject in need thereof, comprising administering a composition provided herein to the subject.

In some aspects, provided herein are methods of increasing meat tenderness in a subject in need thereof, comprising administering a composition provided herein to the subject.

In some aspects, provided herein are methods of reducing meat mass cooking loss in a subject in need thereof, comprising administering a composition provided herein to the subject.

In some aspects, provided herein are methods of improving meat quality in a subject in need thereof, comprising administering a composition provided herein to the subject.

In some aspects, provided herein are methods of treating a viral infection in a subject in need thereof, comprising administering a composition provided herein to the subject.

In some aspects, provided herein are methods of reducing feed cost in raising a subject to maturity, comprising administering a composition provided herein to the subject in need thereof, wherein the subject maintains weight gain consistent with a subject not administered the composition, and both subjects are fed the same feed stock. In some embodiments, the feed cost is reduced up to about 5%, up to about 6.7%, up to about 10%, about 5% to about 10%, about 5% to about 6.7%, or about 6.7% to about 10%.

In some aspects, provided herein are methods of treating a viral infection in a subject in need thereof, comprising administering a composition provided herein to the subject in combination with an antibody therapeutic agent. In some embodiments, the antibody therapeutic agent is a Newcastle disease virus antibody. In some embodiments, the antibody is an avian infectious bronchitis virus antibody. In some embodiments, the antibody is an infectious bursal disease virus. In some embodiments, the viral infection is a Newcastle disease viral infection. In some embodiments, the viral infection is an avian infectious bronchitis viral infection. In some embodiments, the viral infection is an infectious bursal disease viral infection.

In some aspects, provided herein are methods of improving the feed to subject mass conversion ratio (feed conversion efficiency) in a subject in need thereof, comprising administering a composition comprising Formula I to the subject.

In some aspects, provided herein are methods of improving weight gain in a subject in need thereof, comprising administering a composition comprising Formula I to the subject.

In some aspects, provided herein are methods of improving myocardial function in a subject in need thereof, comprising administering a composition comprising Formula I to the subject.

In some aspects, provided herein are methods of decreasing the rate of mortality in a subject in need thereof, comprising administering a composition comprising Formula I to the subject.

In some aspects, provided herein are methods of decreasing the rate of bacterial infection in a subject in need thereof, comprising administering a composition comprising Formula I to the subject.

In some aspects, provided herein are methods of decreasing the rate of diarrhea in a subject in need thereof, comprising administering a composition comprising Formula I to the subject.

In some aspects, provided herein are methods of decreasing the rate of premature death in a subject in need thereof, comprising administering a composition comprising Formula I to the subject.

In some aspects, provided herein are methods of treating a bacterial infection in a subject in need thereof, comprising administering a composition comprising Formula I to the subject.

In some aspects, provided herein are methods of preventing a bacterial infection in a subject in need thereof, comprising administering a composition comprising Formula I to the subject.

In some aspects, provided herein are methods of treating diarrhea in a subject in need thereof, comprising administering a composition comprising Formula I to the subject.

In some aspects, provided herein are methods of treating colibacillosis in a subject in need thereof, comprising administering a composition comprising Formula I to the subject.

In some aspects, provided herein are methods of treating coccidiosis in a subject in need thereof, comprising administering a composition comprising Formula I to the subject.

In some aspects, provided herein are methods of treating swine dysentery in a subject in need thereof, comprising administering a composition comprising Formula I to the subject.

In some aspects, provided herein are methods of preventing diarrhea in a subject in need thereof, comprising administering a composition comprising Formula I to the subject.

In some aspects, provided herein are methods of treating liver disease in a subject in need thereof, comprising administering a composition comprising Formula I to the subject.

In some aspects, provided herein are methods of treating immune disease in a subject in need thereof, comprising administering a composition comprising Formula I to the subject.

In some aspects, provided herein are methods of improving the immune system in a subject in need thereof, comprising administering a composition comprising Formula I to the subject.

In some aspects, provided herein are methods of increasing daily weight gain in a subject in need thereof, comprising administering a composition comprising Formula I to the subject.

In some aspects, provided herein are methods of increasing meat tenderness in a subject in need thereof, comprising administering a composition comprising Formula I to the subject.

In some aspects, provided herein are methods of reducing meat mass cooking loss in a subject in need thereof, comprising administering a composition comprising Formula I to the subject.

In some aspects, provided herein are methods of improving meat quality in a subject in need thereof, comprising administering a composition comprising Formula I to the subject.

In some aspects, provided herein are methods of treating a viral infection in a subject in need thereof, comprising administering a composition comprising Formula I to the subject.

In some aspects, provided herein are methods of reducing feed cost in raising a subject to maturity, comprising administering a composition comprising Formula I to the subject in need thereof, wherein the subject maintains weight gain consistent with a subject not administered the composition, and both subjects are fed the same feed stock.

In some aspects, provided herein are methods of treating a Newcastle disease viral infection in a subject in need thereof, comprising administering a composition comprising Formula I to the subject in combination with a Newcastle disease virus antibody.

In some aspects, provided herein are methods of treating an avian infectious bronchitis viral infection in a subject in need thereof, comprising administering a composition comprising Formula I to the subject in combination with an avian infectious bronchitis virus antibody.

In some aspects, provided herein are methods of treating an infectious bursal disease viral infection in a subject in need thereof, comprising administering a composition comprising Formula I to the subject in combination with an infectious bursal disease virus antibody.

In some aspects, provided herein are methods of improving the feed to subject mass conversion ratio (feed conversion efficiency) in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of improving weight gain in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of improving myocardial function in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of decreasing the rate of mortality in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of decreasing the rate of bacterial infection in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of decreasing the rate of diarrhea in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of decreasing the rate of premature death in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of treating a bacterial infection in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of preventing a bacterial infection in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of treating diarrhea in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of treating colibacillosis in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of treating coccidiosis in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of treating swine dysentery in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of preventing diarrhea in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of treating liver disease in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of treating immune disease in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of improving the immune system in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of increasing daily weight gain in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of increasing meat tenderness in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of reducing meat mass cooking loss in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of improving meat quality in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of treating a viral infection in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%.

In some aspects, provided herein are methods of reducing feed cost in raising a subject to maturity, comprising administering to the subject in need thereof a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%, wherein the subject maintains weight gain consistent with a subject not administered the composition, and both subjects are fed the same feed stock.

In some aspects, provided herein are methods of treating a Newcastle disease viral infection in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%, in combination with a Newcastle disease virus antibody.

In some aspects, provided herein are methods of treating an avian infectious bronchitis viral infection in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%, in combination with an avian infectious bronchitis virus antibody.

In some aspects, provided herein are methods of treating an infectious bursal disease viral infection in a subject in need thereof, comprising administering to the subject a composition comprising: about 19.6 A_{GC}% α-terpinolene; about 52.5 A_{GC}% terpinen-4-ol; about 9.2 A_{GC}% α-terpineol; about 1.8 A_{GC}% 1,8-cineole; about 2.2 A_{GC}% γ-terpinene; α-pinene about 1.6 A_{GC}%; sabinene up to about 0.01 A_{GC}%; α-terpinene up to about 0.01 A_{GC}%; limonene up to about 0.01 A_{GC}%; p-cymene up to about 0.01 A_{GC}%; aromadendrene up to about 0.01 A_{GC}%; ledene up to about 0.01 A_{GC}%; δ-cadinene up to about 0.01 A_{GC}%; globulol up to about 0.01 A_{GC}%; and virdiflorol up to about 0.01 A_{GC}%, in combination with an infectious bursal disease virus antibody.

In some aspects, provided herein are methods of purifying a Melaleuca alternifolia extract, wherein the purified extract is a composition provided herein. Thus, also provided herein is a purified M. alternifolia extract.

In some embodiments, the M. alternifolia extract is an oil. In some embodiments, the purified M. alternifolia extract is an oil.

In some embodiments, the M. alternifolia extract is an oil that conforms to International standard ISO 4730:2004 or ISO 4730:2017.

In some embodiments, the M. alternifolia extract is an oil that is extracted from M. alternifolia plants by steam distillation.

In some embodiments, the M. alternifolia extract is purified by gas flushing (sparging) distillation.

In some embodiments of the methods provided herein, the subject is a clade. In some embodiments, the subject is a clade of livestock. In some embodiments, the subject is a livestock. In some embodiments, the subject is a population of subjects. In some embodiments, the subject is a population of livestock. In some embodiments, the livestock is anatine, bovine, caprine, cervine, equine, galline, glirine, leporine, meleagrine, ovine, porcine, rangiferine, ratite, suilline, taurine, vituline, zebu, or a combination thereof. In some embodiments, the subject is a human being. In some embodiments, the subject is a mammal. In some embodiments, the subject is a poultry.

In some embodiments of the methods provided herein, the composition is administered systemically.

In some embodiments of the methods provided herein, the composition is administered by enteral administration. In some embodiments, the composition is administered by oral administration, gastric feeding tube, duodenal feeding tube, gastrostomy, rectal administration, or vaginal administration. In some embodiments, the composition is administered by oral administration.

In some embodiments of the methods provided herein, the composition is administered by parenteral administration. In some embodiments, the composition is administered by intramuscular administration, subcutaneous administration, intravenous administration, intradermal administration, intra-arterial administration, or intraosseous administration.

In some embodiments of the methods provided herein, the composition is administered topically.

In some embodiments of the methods provided herein, the composition is administered by epicutaneous administration, inhalation, enema, ophthalmic administration, otic administration, or nasal administration.

In some embodiments of the methods provided herein, the methods provided above further comprise administration to the subject at least one additional therapeutic agent (e.g., at least one antibiotic agent or at least one antiviral agent).

In some embodiments of the methods provided herein, the composition is administered in a therapeutically effective amount.

### Formulations

In some aspects, provided herein is a pharmaceutical composition comprising a composition provided herein, together with a pharmaceutically acceptable excipient.

In some aspects, provided herein is a dosage unit form, comprising a composition provided herein. In some embodiments, the dosage unit form is suitable for administration to the subject.

Actual dosage levels of the active ingredients in the pharmaceutical compositions provided herein may be varied so as to obtain an amount of the active ingredients that is effective to achieve the desired therapeutic response for a particular subject, composition, and mode of administration, without being toxic to the subject.

In particular, a selected dosage level will depend upon a variety of factors including activity of the particular composition employed, time of administration, rate of excretion of the compounds of the composition, duration of the treatment, other drugs, compounds or materials used in combination with the composition, age, sex, weight, condition, general health, and prior medical history of the patient being treated, and like factors well-known in the medical arts.

A medical doctor, e.g., physician or veterinarian, having ordinary skill in the art may readily determine and prescribe an effective amount of the pharmaceutical composition required. For example, the physician or veterinarian could start doses of the pharmaceutical compositions provided herein at levels lower than that required in order to achieve the desired therapeutic effect and gradually increase the dosage until the desired effect is achieved.

In particular embodiments, it is especially advantageous to formulate the composition in dosage unit form for ease of administration and uniformity of dosage. Dosage unit form as used herein refers to physically discrete units suited as unitary dosages for the patients to be treated—each unit containing a predetermined quantity of therapeutic compound calculated to produce the desired therapeutic effect in association with the required pharmaceutical vehicle. The dosage unit forms herein are dictated by and directly dependent on (a) the unique characteristics of the therapeutic compounds of the composition and the particular therapeutic effect to be achieved, and (b) the limitations inherent in the art of compounding or formulating such a therapeutic composition for the methods provided herein.

In some embodiments, the compositions provided herein are formulated using one or more pharmaceutically acceptable excipients or carriers. In some embodiments, the pharmaceutical compositions provided herein comprise a therapeutically effective amount of a composition provided herein and a pharmaceutically acceptable excipient.

Compositions provided herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, about 5 mg, about 10 mg, about 20 mg, about 30 mg, about 40 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000mg, or any and all whole or partial increments there between, or a range bounded by any two of these values.

In some embodiments, the dose of a composition provided herein is from about 1 mg to about 2,500 mg. In some embodiments, a dose of a composition provided herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg, about 5 mg, about 10 mg, about 20 mg, about 30 mg, about 40 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000mg, or a range bounded by any two of these values.

In some embodiments, a dose of a second compound (i.e., another therapeutic agent useful for the methods provided herein) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof, or a range bounded by any two of these values.

In some embodiment, provided herein is a packaged pharmaceutical composition comprising a container holding a therapeutically effective amount of a composition provided herein, alone or in combination with a second pharmaceutical agent or composition; and instructions for using the composition according to a method provided herein.

In some embodiments, provided is a kit comprising a composition provided herein, packaging, and instructions for use of the composition.

Routes of administration of any of the compositions provided herein include, in some embodiments, oral, nasal, rectal, intravaginal, parenteral, buccal, sublingual or topical. The compositions for use as described herein may be formulated for administration by any suitable route, such as for oral or parenteral, for example, transdermal, transmucosal (e.g., sublingual, lingual, (trans)buccal, (trans)urethral, vaginal (e.g., trans- and peri-vaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, or topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful as provided herein are not limited to the particular formulations and compositions that are described herein.

For oral application, particularly suitable are tablets, dragees, liquids, drops, suppositories, capsules, caplets, or gelcaps. The compositions intended for oral use may be prepared according to any method known in the art, and such compositions may contain one or more agents selected from the group consisting of inert, non-toxic pharmaceutical excipients that are suitable for the manufacture of tablets. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the compositions provided herein may be formulated for injection or infusion, for example, intravenous, intramuscular or subcutaneous injection or infusion, or for administration in a bolus dose, continuous infusion, or both. Suspensions, solutions, or emulsions in an oily or aqueous vehicle, optionally containing other formulatory agents such as suspending, stabilizing, dispersing agents, or a combination thereof may be used.

Those skilled in the art will recognize, or be able to ascertain using no more than routine experimentation, numerous equivalents to the specific procedures, embodiments, claims, and examples described herein.

It is to be understood that wherever values and ranges are provided herein, all values and ranges encompassed by these values and ranges, or a combination of these values and ranges, are meant to be encompassed within the scope of the aspects and embodiments provided herein. Moreover, all values that fall within these ranges, as well as the upper or lower limits of a range of values, are also contemplated by the present application.

### EXAMPLES

The following Examples further illustrate aspects of the compositions and methods provided herein. However, these Examples are in no way a limitation of the teachings or disclosure as set forth herein. These Examples are provided for illustration purposes.

### Example 1

### Preparation of Formula I in Various Forms

The term “monoterpene” refers to compounds derived from an isomer unit that has the formula C₁₀H₁₆ and C₁₀H₁₈O. Monoterpenes found in the essential oil from Melaleuca alternifolia include α-pinene, α-thujene, β-pinene, sabinene, α-phellandrene, α-terpinene, limonene, p-cymene, γ-terpinene, terpinolene, terpenin-4-ol, α-terpineol, aromadendren, ledene, δ-cadinene, globulol, and virdiflorol.

The term “conventional Melaleuca alternifolia oil” refers to oil extracted from a Melaleuca plant that complies with ISO 4730:2004 and AS 2782-2009. Preferably, the oil is extracted from Melaleuca alternifolia.

Essential oils extracted from Melaleuca species comprise a monoterpene fraction, an oxygenated monoterpene fraction, and a sesquiterpene fraction, albeit the amount is variable in each fraction depending on the species from which the oil is extracted. Of these fractions, some of the monoterpenes are generally more volatile and have a lower relative molecular weight. Thus, they may be removed by techniques such as sparging, fractional distillation, chromatographic techniques, and selective solvent extraction techniques. The monoterpenes found in Melaleuca oil have boiling points ranging from about 155° C. for α-pinene to about 185° C. for terpinolene. Terpinen-4-ol has a boiling point of 212° C. The sesquiterpenes aromadendrene and alloaromadendrene have boiling points of 257° C. to 258° C. The differences in these boiling points can be leveraged by the technique of fractional distillation. Chromatographic techniques have been described in U.S. Pat. No. 4,605,783, and also by Hayashi et al. (Bulletin of the Chemical Society of Japan, 1969, vol. 42, p. 3026-3028). Nanofiltration is a relatively recent membrane filtration process used on natural organic matter and synthetic organic matter.

Gas flushed distillation is used herein to isolate certain oil fractions of a complex mixture of oils (e.g., conventional Melaleuca alternifolia oil), sparging flushed distillation is optionally used in conjunction with, or separate from, gas flushed distillation. In some embodiments, preferred parameters for gas flushed distillation are: pressure and temperature for extraction of 90 psi and 65° C., respectively, and sparging flush at 5000 mL/min, inert gas flow at 20 mL/min add to control buffer reaction. Using the techniques herein, between about 15% and about 28% of the monoterpenes are removed—typically around 18% to 25% (e.g., around 20%). This will achieve C₁₀H₁₆ and C₁₀H₁₈O fractions of over 78 A_{GC}%, preferably terpinolene together with terpenin-4-ol of over 72 A_{GC}%.

It may be seen that the combined minimum and maximum contents for all of the monoterpenes in Melaleuca oil defined by both the Australian and International standards provides a range of monoterpene content. It may therefore be clearly appreciated that the compositions provided herein comprise individual monoterpene content falling well outside the standard range, and therefore the compositions provided herein cannot be considered to have the same chemical profile as conventional Melaleuca or tea tree oil.

In some embodiments provided herein, a composition is derived from the essential oil of Melaleuca alternifolia and typically comprises terpinolene and terpinen-4-ol from between about 40% to about 80 A_{GC}%, between about 65% to about 75 A_{GC}%, or between about 72% to about 78 A_{GC}%, and α-terpineol between about 3 to about 11 A_{GC}%, between about 4% to about 9 A_{GC}%, or between about 5% to about 8 A_{GC}%. These ranges include intermediate subranges and values, and these fractions include or do not include α-pinene, sabinene, α-terpinene, limonene, p-cymene, 1,8-cineole, γ-terpinene, aromadendrene, viridiflorene, delta cadinene, globulol, viridiflorol, or combinations thereof.

TABLE 2
comparison of conventional Melaleuca oil (ISO 4730:2004, AS 2782-2009)
to Formula I.

| | ISO 4730:2004 | | Formula I |
|---|---|---|---|
| Component | Minimum A_{GC} % | Maximum A_{GC} % | A_{GC} % |
| α-Pinene | 1.00 | 6.00 | 1.60 |
| Sabinene | trace | 3.50 | trace |
| α-Terpinene | 5.00 | 13.00 | trace |
| Limonene | 0.50 | 1.50 | trace |
| p-Cymene | 0.50 | 8.00 | trace |
| 1,8-Cineole | trace | 15.00 | 1.80 |
| γ-Terpinene | 10.00 | 28.00 | 2.20 |
| Terpinolene | 1.50 | 5.00 | 19.6 |
| Terpinen-4-ol | 30.00 | 48.00 | 52.5 |
| α-Terpineol | 1.50 | 8.00 | 9.2 |
| Aromadendrene | trace | 3.00 | trace |
| Ledene | trace | 3.00 | trace |
| δ-Cadinene | trace | 3.00 | trace |
| Globulol | trace | 1.00 | trace |
| Viridiflorol | trace | 1.00 | trace |

The compositions provided herein may be in any suitable form such as in solid form preparations (e.g., powders, tablets, dispersible granules, capsules, cachets, or suppositories), or in liquid form preparations (e.g., solutions, suspensions, or emulsions, which are, e.g., suitable for oral ingestion). In some embodiments, the compositions are stored protected from ultraviolet irradiation (e.g., protected from sunlight), and at standard temperature and pressure.

An exemplary composition (Formula I) was prepared from tea tree oil (see Table 2). In some embodiments, the compositions provided herein are formed by combining the specified amounts of purified components (e.g., commercial grade) of the compositions.

To obtain a solid form preparation, a pharmaceutically acceptable excipient (e.g., microcrystalline cellulose) is used as an intermediate by mixing the excipient (e.g., microcrystalline cellulose) with Formula I at a ratio of 4:1 at 40° C. in a rotary oven over two hours. A 2% vol/vol liquid suspension of Formula I is made with a mixture of 4% mass/mass of d-tocopherol polyethylene glycol 1000 succinate (TPGS-1000) in water (e.g., 4 g TPGS-1000/1000 mL water) well dispersed at 90° C., which mixture is combined with Formula Ito make the 2% vol/vol liquid suspension of Formula I in the TPGS-1000 mixture.

### Example 2

### Formula I Prepared for LD₅₀ Toxicity Test

The aim of the toxicity study is to evaluate the acute oral toxicity and LD₅₀ value of Formula I on ICR mice and Albino Wistar rat.

Methods: Food grade microcrystalline cellulose was combined with Formula I in a 75:25 ratio, and mixed at 40° C. in a rotary oven over two hours.

A group of 10 male and 10 female ICR mice and a group of 10 male and 10 female Albino Wistar rats were drawn at age 6 weeks old. Animals were ranked by weight and sex and then randomly allocated into four groups. Two groups of females (n₁=10, n₂=10) and two groups of males (n₁=10, n₂=10). The treatment groups met the regulatory requirements of having the No Observable Effects Limit (“NOEL”) group, the Lowest Observable Effects Limit (“LOEL”) group, and the Mean Dose Lethality (MDL) or Maximum Toxicity Dose (“MTD”) group.

During a pre-test of MTD intragastrical administration, no sign of any negative effect was seen in the animals. Intragastric administration of 5000 mg/kg.b.w. (25% Formula I) over a period of 14 days for each group—the test showed that mortality was nil during the test period. There were no signs of intoxication. No animals were observed having loss of diet/appetite, respiration difficulty, sedation, loss of locomotor activity, drowsiness, or lacrimation during the test period. All animals appeared healthy through the 14 day test period. None of the animals in the test dose groups had been negatively affected, and therefore it was determined that there was no toxic effect when 5000 mg/kg.b.w. (25% Formula I) was administered as an acute study for toxicity. The absence of observed toxicity suggests that Formula I had no adverse effect on the subjects.

### Example 3

### Effect of Formula I on Weaner Piglets

Weaner piglet diarrhoea rate has the largest effect on weaner piglet productivity. The aim of this study was to examine the influence of Formula I on weaner piglet diarrhea, which stops piglet growth and severely affects grower-pig productivity. Also, the influence of Formula I on weaner piglet biochemical profile and intestinal system was examined in order to determine how Formula I will affect the immune system.

Feed preparation: standard feed and feed nutrition, standard weaner piglet farming management, 21-day trial period, free water and feed five times a day at 07:00, 10:00, 14:00, 17:00 and 19:00 daily.

Food grade microcrystalline cellulose and Formula I were well mixed at room temperature at a ratio of 80:20 by weight—referred to herein as MA.

Weaner piglets trial preparation: 90 head of similar weight (7.0±0.5 kg), similar age (21±3 days) three generation cross-breeding weaner piglets were chosen, then randomly divided into five testing groups, each group had three repeats, each repeat had six piglets (half male and half female). All groups had standard feed and feed nutrition.

Group Configuration:

CON—control group, standard weaner piglets feed (no antibiotics or Formula I (e.g., MA));

ANT—antibiotics group, standard weaner piglets feed, 200 mg/kg of 10% antibiotics colistin and 75 mg/kg of 15% of chlortetracycline per 1 kg feed;

L-MA—low-MA group, 250 mg MA per 1 kg feed;

M-MA—mid-MA group, 500 mg MA per 1 kg feed; and

H-MA—high-MA group, 750 mg MA per 1 kg feed.

Standard formulae were used to calculate piglet diarrhea rate and index, daily weight and final weight. Standard processes of humane slaughter, serum collection, and standard ELISA kits for all related profile test were used.

TABLE 3a
Effect of Formula I on weaner piglet productivity patameters.

| Group | CON | ANT | L-MA | M-MA | H-MA | σ | P |
|---|---|---|---|---|---|---|---|
| ADG (g) | 151.43 | 174.43 | 140.58 | 160.77* | 160.77* | 7.65 | 0.76-7 |
| ADFI (g) | 258.04 | 258.68 | 250.79 | 257.14 | 275.45 | 6.39 | 0.82-3 |
| FCR | 1.79 | 1.47 | 1.82 | 1.61** | 1.72* | 0.06 | 0.42-5 |
| Diarrhea rate (DR) | 8.73 | 6.88 | 9.90 | 4.23** | 6.34* | 1.32 | 0.46-5 |
| Diarrhea index (DI) | 4.56 | 3.22 | 5.28 | 2.44** | 3.72* | 0.65 | 0.44-4 |

ADG - average daily gain,
ADFI - average daily feed intake,
FCR - feed conversation rate,
*significant difference,
**extreme difference.

Results (Table 3a) showed that the M-MA group had a 10.05% better FCR compared to the control group and also had the lowest DR (51.55% better than CON) and DI. Diarrhoea is one of the biggest problems in pig farming with colibacillosis, coccidiosis, and swine dysentery as the leading contributors to weaner piglet death. Diarrhea, if not treated, may well lead to the death of the entire litter via contamination. Taking into account the mortality rate caused from diarrhea and FCR, the M-MA group would yield the overall highest return and offer the best animal welfare.

TABLE 3b
Formula I effect on weaner piglet serum biochemical profile.

| Group | CON | ANT | L-MA | M-MA | H-MA | σ | P |
|---|---|---|---|---|---|---|---|
| ALT (U/L) | 90.83 | 88.83 | 74.67 | 78.50 | 70.33 | 5.17 | 0.696 |
| AST (U/L) | 187.33 | 188.83 | 108.00* | 132.50* | 99.83** | 18.38 | 0.383 |
| ALP (U/L) | 483.17 | 397.00 | 467.67 | 550.00 | 443.67 | 23.53 | 0.351 |
| GGT (U/L) | 117.67 | 56.33 | 51.00* | 41.50** | 41.50** | 13.52 | 0.360 |
| TBIL (μmol/L) | 0.52 | 0.42 | 0.48 | 0.63 | 0.72 | 0.56 | 0.468 |
| TP (g/L) | 51.05 | 49.52 | 50.17 | 48.08 | 47.63 | 0.73 | 0.576 |
| ALB (g/L) | 26.12 | 26.13 | 26.40 | 25.90 | 25.10 | 0.42 | 0.905 |
| GLO (g/L) | 24.93 | 23.38 | 23.77 | 22.30 | 22.53 | 0.59 | 0.666 |
| A/G | 1.07 | 1.13 | 1.12 | 1.20 | 1.14 | 0.04 | 0.898 |
| TBA (μmol/L) | 19.55 | 34.52** | 43.73** | 52.93* | 19.37 | 4.09 | 0.02 |
| UREA (mmol/L) | 3.10 | 3.03 | 4.28 | 3.53 | 3.05 | 0.19 | 0.191 |
| CRE (μmol/L) | 67.00 | 71.67 | 66.00 | 65.50 | 63.17 | 1.53 | 0.529 |
| LDH (U/L) | 1267.50 | 1252.67 | 1237.00 | 1266.50 | 1065.50 | 70.57 | 0.896 |
| TG (mmol/L) | 0.50 | 0.63 | 0.52 | 0.60 | 0.48 | 0.03 | 0.466 |
| TC (mmol/L) | 2.04 | 2.02 | 1.78 | 2.03 | 1.94 | 0.04 | 0.295 |
| HDL-C (mmol/L) | 0.58 | 0.58 | 0.48 | 0.57 | 0.56 | 0.02 | 0.508 |
| LDL-C (mmol/L) | 0.97 | 1.01 | 0.91 | 1.02 | 0.97 | 0.03 | 0.698 |
| GLU (mmol/L) | 5.25 | 5.94 | 4.46** | 5.15* | 4.63** | 0.20 | 0.143 |
| CHE (U/L) | 638.67 | 696.33 | 714.33 | 741.17 | 646.17 | 18.84 | 0.373 |

ALT - Alanine aminotransferase,
AST - Aspartate transaminase,
ALP - Alkaline phosphatase,
GGT - l-γ-glutamyl transferase,
TBIL - total bilirubin,
TP - total protein,
ALB - albumin,
GLO - globulin,
A/G - albumin/globulin,
TBA - total bile acids,
UREA - serum urea,
CRE - creatinine,
LDH - lactate dehydrogenase,
TG - triglyceride,
TC - total cholesterol,
HDL-C - high density lipoproteins,
LDL-C - low density lipoproteins,
GLU - glucose,
CHE - cholinesterase,
*significant difference,
**extreme difference.

From Table 3b, compared with the CON group, LM and MM group serum bile acid contents increased by 144% and 222% respectively, the MM group increased significantly (P<0.05). Compared with the CON group, L-MA, M-MA and H-MA group, aspartate aminotransferase was reduced by 42.34%, 29.27% and 46.71% respectively. Compared with the CON group, L-MA, M-MA and H-MA group, serum l-γ-glutamyl transferase was reduced by 66.67%, 64.73% and 64.73% respectively.

Bile acid in liver cells is derived from cholesterol, bile acid absorption of fat and fat-soluble vitamins, and plays an important role in transport and distribution functions. Bile acid can also act as a nuclear receptor signal molecule which activates and regulates cholesterol metabolism. Serum bile acid elevation also plays important physical roles in the lately pig farming productivity. Formula I has demonstrated lately in grower-pig farming, which not only increasing pig farming productivity, also have achieved batter feed conversion rates, grain weight and improve quality of meats.

Cardiac muscle contains more aspartate aminotransferase than any other organ. When cardiac muscle bolt death occurs aspartate aminotransferase enzyme activity increased significantly. Formula I reduced the activity of the aspartate aminotransferase which shows Formula I plays a certain role in improving myocardial function.

l-γ-glutamyl transferase, mainly from liver system, is used as an indicator of liver system diseases. Diseases such as hepatitis and cholecystitis often cause the rise of l-γ-glutamyl transferase. Formula 1 reduced l-γ-glutamyl transferase activity which shows that it has certain protective effects on the liver system.

TABLE 3c
influence of Formula I on weaner piglet organ index.

| Group | CON | ANT | L-MA | M-MA | H-MA | σ | P |
|---|---|---|---|---|---|---|---|
| heart | 5.25 | 5.09 | 5.25 | 4.86 | 5.19 | 0.08 | 0.486 |
| liver | 26.12 | 24.02 | 26.40* | 27.36* | 28.38* | 0.45 | 0.022 |
| spleen | 2.41 | 2.06 | 2.10 | 2.35 | 2.21 | 0.08 | 0.0601 |
| lung | 16.70 | 10.30* | 12.20* | 11.62* | 12.35* | 0.61 | 0.005 |
| kidney | 6.08 | 5.95 | 6.64 | 6.04 | 5.54 | 0.14 | 0.386 |
| thymus | 0.94 | 0.78 | 1.52* | 0.95 | 1.01* | 0.09 | 0.058 |
| pancreas | 2.30 | 1.87 | 2.07 | 2.10 | 1.72 | 0.12 | 0.616 |

*significant difference.

The results summarized in Table 3c show that usage of antibiotics has a detrimental effect on all the organ indices. This indicates inferior organ functions relative to the body mass. Notably, L-MA has a remarkable thymus organ index which plays a vital role in the immune system.

TABLE 3d
influence of Formula I on weaner piglet intestine relative weight.

| Group | CON | ANT | L-MA | M-MA | H-MA | σ | P |
|---|---|---|---|---|---|---|---|
| duodenum | 1.64 | 1.57 | 1.74 | 1.81 | 1.20 | 0.10 | 0.333 |
| jejunum | 21.47 | 21.97 | 23.93 | 23.88 | 22.04 | 0.44 | 0.024 |
| ileum | 22.39 | 22.97 | 20.71 | 22.20 | 21.09 | 0.53 | 0.724 |
| colon | 13.58 | 10.82* | 13.39 | 13.25 | 12.02 | 0.34 | 0.033 |
| cecum | 2.39 | 2.43 | 2.38 | 2.27 | 2.51 | 0.08 | 0.908 |

*significant difference.

From Table 3d, no significant difference (P>0.05) was observed between the duodenum, jejunum, ileum, and cecum of each group. Compared with the CON group, the ANT group colon relative weight was reduced by 2.32%, which was a statistically significant level (P<0.05).

TABLE 3e
influence of Formula I on weaner piglet intestines
per unit length of weight.

| Group | CON | ANT | L-MA | M-MA | H-MA | σ | P |
|---|---|---|---|---|---|---|---|
| duodenum | 6.94 | 6.89 | 6.12 | 7.12 | 5.25 | 0.33 | 0.361 |
| jejunum | 2.47 | 2.26 | 3.05* | 2.53 | 2.59 | 0.07 | 0.007 |
| colon | 8.26 | 7.31 | 9.29** | 11.99** | 8.59* | 0.67 | 0.234 |
| cecum | 22.12 | 19.95 | 21.14 | 20.04 | 23.83 | 0.79 | 0.508 |

*significant difference,
**extreme difference.

From Table 3e, duodenum and cecum per unit length between groups was not significantly different (P>0.05). Compared with CON group, L-MA, M-MA, and H-MA group jejunum unit length weight increased by 23.48%, 23.48%, and 2.43%, respectively, and, L-MA group reached significant level (P<0.05).

In conclusion, Formula I (e.g., MA) application (e.g., about 250 mg, about 500 mg, about 750 mg, about 250 mg to about 500 mg, about 500 mg to about 750 mg, or about 250 mg to about 750 mg per kg feed) improved the weaner piglet diarrhea rate, improved productivity performance and blood biochemical profile, effectively enhanced the pig's immune ability, regulated lipid metabolism, and improved the function of myocardium and liver system. In other words, administering about 65 mg, about 125 mg, about 190 mg, about 65 mg to about 125 mg, about 125 mg to about 190 mg, or about 65 mg to about 190 mg of Formula I (e.g., MA) to a weaner piglet improved the weaner piglet diarrhea rate, improved productivity performance and blood biochemical profile, effectively enhanced the pig's immune ability, regulated lipid metabolism, and improved the function of myocardium and liver system.

### Example 4

### Formula I Influence on Grower-Pig Farming Productivity

The aim of the study was to carry out an evaluation of Formula I in grower-pig farming productivity, mortality rate, biochemical profile and meat quality.

Grower-pig trial preparation: 56 head of Duroc cross-breeding (male only) were chosen, similar weight (68 kg±1.5 kg), similar age, randomly divided into four testing groups, each group has four repeats, each repeat has four heads on 56 days trail.

Feed configuration: all groups had basic standard grower-pig feed.

Food grade microcrystalline cellulose and Formula I were well mixed at room temperature at a ratio of 80:20 by weight—referred to herein as MA.

Group Configuration:

CON—control group, standard grower-pig feed (no antibiotics or Formula I (e.g., MA));

L-MA—low-MA group, 100 mg MA per 1 kg grower-pig feed;

M-MA—mid-MA group, 200 mg MA per 1 kg grower-pig feed; and

H-MA—high-MA group, 300 mg MA per 1 kg grower-pig feed.

Standard grower-pig farming management was used: 56-day trial, free water and feed twice a day between 7:00-8:00 and 16:30-17:30. Standard testing formulae were used to calculate grower-pig productivity, daily weight and final weight.

Standard processes of humane slaughter and serum collection were used. Standard processes to analyze serum biochemical profile, and meat quality. Standard ELISA kits were used for all related profile testing.

TABLE 4a
Influence of Formula I on grower-pig productivity.

| | CON | L-MA | M-MA | H-MA |
|---|---|---|---|---|
| Starting pig weight (kg) | 67.94 ± 0.26 | 68.31 ± 0.16 | 68.25 ± 0.23 | 68.00 ± 0.10 |
| Finishing pig weight (kg) | 114.29 ± 0.93 | 119.04 ± 1.84* | 119.81 ± 1.58* | 119.56 ± 2.01* |
| ADG (g) | 827.75 ± 17.86 | 905.88 ± 34.00* | 920.76 ± 30.08** | 920.76 ± 35.41** |
| ADFI | 2.95 ± 0.07 | 2.91 ± 0.02 | 3.02 ± 0.06 | 3.04 ± 0.04 |
| (kg) | | | | |
| FIG | 3.57 ± 0.13 | 3.23 ± 0.13* | 3.28 ± 0.04* | 3.31 ± 0.10* |

ADG—average daily grain, ADFI—average daily feed intake, F/G—feed/weight-gain, *significant difference, **extreme difference.

Table 4a shows that, compared with the CON group, the pig weights in the L-MA, M-MA, and H-MA groups significantly increased. The average pig weight in the L-MA, M-MA, and H-MA groups increased by 4.16%, 4.82%, and 4.61%, respectively.

Compared to the CON group, the M-MA and H-MA groups average daily weight gain (ADG) increased by 11.24%, which is significantly higher than the control group (P<0.05).

In conclusion, when compared to the CON group, L-MA and M-MA had significant weight gain and feed conversion efficiency up to 10.53% and 8.84%. Supplementing standard grower-pig feed with about 100 mg to about 200 mg, or about 300 mg (e.g., about 100 to about 200, about 200 to about 300, or about 100 to about 300 mg) of Formula I (e.g., MA) improved grower-pig weight gain, and feed conversion efficiency. In other words, administering about 300 mg, about 600 mg, about 900 mg, about 300 mg to about 600 mg, about 600 mg to about 900 mg, or about 300 mg to about 900 mg of Formula I (e.g., MA) to a grower-pig improved grower-pig weight gain, and feed conversion efficiency.

TABLE 4b
influence of Formula I on grower-pig mortality rate.

| Group | Mortality | Rate |
|---|---|---|
| CON | 2 | 12.5% |
| L-MA | 1 | 6.25% |
| M-MA | 0 | 0 |
| H-MA | 0 | 0 |

In conclusion, all Formula I (MA) groups had a significantly lower mortality rate than the control group. Supplementing standard grower-pig feed with about 100 mg to about 200 mg, or about 300 mg (e.g., about 100 to about 200, about 200 to about 300, or about 100 to about 300 mg) of Formula I (e.g., MA) improved grower-pig mortality rate. In other words, administering about 300 mg, about 600 mg, about 900 mg, about 300 mg to about 600 mg, about 600 mg to about 900 mg, or about 300 mg to about 900 mg of Formula I (e.g., MA) to a grower-pig improved grower-pig mortality rate.

TABLE 4c
effect of Formula I on grower-pig serum biochemical profile.

| Group | CON | L-MA | M-MA | H-MA |
|---|---|---|---|---|
| ALT | 50.50 ± 3.87 | 56.17 ± 3.90 | 51.33 ± 2.40 | 54.33 ± 4.14 |
| AST | 49.00 ± 4.67 | 54.67 ± 1.98 | 39.80 ± 2.23* | 36.20 ± 1.98** |
| AST/ALT | 0.91 ± 0.07 | 1.00 ± 0.08* | 0.81 ± 0.07* | 0.72 ± 0.06* |
| TP | 68.32 ± 1.63 | 72.57 ± 1.15* | 67.68 ± 1.67 | 70.85 ± 0.92* |
| ALB | 35.52 ± 0.52 | 35.20 ± 1.02 | 35.43 ± 1.32 | 37.92 ± 1.04 |
| GLO | 31.80 ± 1.62 | 37.37 ± 1.25** | 32.25 ± 1.69* | 32.93 ± 1.30* |
| ALB/GLO | 1.17 ± 0.06 | 0.95 ± 0.05* | 1.12 ± 0.09 | 1.17 ± 0.07 |
| ALP | 122.67 ± 8.23 | 148.50 ± 14.41 | 143.17 ± 10.62 | 138.00 ± 8.08 |
| GGT | 33.00 ± 2.80 | 34.17 ± 4.39 | 36.00 ± 1.71 | 36.17 ± 1.92 |
| LDH | 446.80 ± 20.15 | 482.50 ± 21.23 | 438.50 ± 17.95 | 390.20 ± 14.85 |
| CHE | 573.33 ± 51.07 | 617.67 ± 36.83** | 603.50 ± 21.54** | 677.50 ± 13.76** |
| CRE | 158.67 ± 1.91 | 147.50 ± 5.08** | 145.50 ± 3.26** | 147.83 ± 6.51** |
| BUN | 7.32 ± 0.61 | 8.28 ± 0.63 | 8.37 ± 0.68 | 7.42 ± 0.59 |
| GLU | 5.15 ± 0.47 | 6.55 ± 0.78 | 8.37 ± 0.35 | 7.42 ± 0.11 |
| TC | 2.14 ± 0.05 | 2.24 ± 0.08 | 2.23 ± 0.09 | 2.17 ± 0.09 |
| TG | 0.38 ± 0.02 | 0.52 ± 0.07** | 0.58 ± 0.08** | 0.65 ± 0.07* |
| HDL-C | 0.85 ± 0.04 | 0.92 ± 0.05 | 0.90 ± 0.05 | 0.85 ± 0.04 |
| LDL-C | 1.07 ± 0.05 | 1.10 ± 0.05 | 1.02 ± 0.04 | 1.00 ± 0.07 |
| CPK | 3764.50 ± 124.90 | 3725.83 ± 93.35 | 3595.17 ± 97.89 | 3762.67 ± 184.26 |

ALT—Alanine aminotransferase, AST—Aspartate transaminase, AST/AL—Aspartate transaminase/Alanine aminotransferase, TP—total protein, ALB—albumin, GLO—globulin, ALB/GLO—albumin/globulin, ALP—Alkaline phosphatase, GGT—I-γ-glutamyl transferase, LDH—lactate dehydrogenase, CHE—cholinesterase, CRE—creatinine, BUN—blood urea nitrogen, GLU— glucose, TC—total cholesterol, TG—triglyceride, HDL-C—high density lipoproteins, LDL-C— low density lipoproteins, CPK—creatine phosphate kinase, *significant difference, **extreme difference.

Normally, aspartate transaminase activity is very low. High activity of aspartate transaminase is directly related to the damage of liver cells and mitochondria. In this study, aspartate transaminase activity was significantly reduced in Formula I containing groups compared to the control group, which indicates that Formula I confers some protection to the liver system. Supplementing standard grower-pig feed with about 100 mg to about 200 mg, or about 300 mg (e.g., about 100 to about 200, about 200 to about 300, or about 100 to about 300 mg) of Formula I (e.g., MA) improved grower-pig liver function. In other words, administering about 300 mg, about 600 mg, about 900 mg, about 300 mg to about 600 mg, about 600 mg to about 900 mg, or about 300 mg to about 900 mg of Formula I (e.g., MA) to a grower-pig improved grower-pig liver function.

Globulin mainly reflects the body's immune condition. When compared with the control group, the L-MA group displayed a significant increase in globulin, suggesting Formula I promotes grower-pig immune ability. Supplementing standard grower-pig feed with about 100 mg to about 200 mg, or about 300 mg (e.g., about 100 to about 200, about 200 to about 300, or about 100 to about 300 mg) of Formula I (e.g., MA) improved grower-pig immune function. In other words, administering about 300 mg, about 600 mg, about 900 mg, about 300 mg to about 600 mg, about 600 mg to about 900 mg, or about 300 mg to about 900 mg of Formula I (e.g., MA) to a grower-pig improved grower-pig immune function.

TABLE 4d
impact of Formula I on grower-pig meat quality.

| Group | CON | L-MA | M-MA | H-MA |
|---|---|---|---|---|
| L* | 37.30 ± 1.72 | 37.61 ± 0.75 | 37.57 ± 1.11 | 36.65 ± 0.59 |
| A* | 3.91 ± 0.38 | 4.39 ± 0.48** | 5.28 ± 0.39** | 4.03 ± 0.23* |
| B* | 3.00 ± 0.47 | 3.68 ± 0.27 | 3.56 ± 0.16 | 3.56 ± 0.16 |
| pH₂₄ | 5.68 ± 0.02 | 5.70 ± 0.01* | 5.77 ± 0.02** | 5.72 ± 0.02* |
| Dehydrate | 43.99 ± 1.14 | 42.01 ± 0.47 | 42.77 ± 1.09 | 41.48 ± 0.52 |
| rate | | | | |
| Shear | 2.82 ± 0.07 | 2.45 ± 0.08* | 2.50 ± 0.08** | 2.69 ± 0.10* |
| force | | | | |
| Drip loss | 3.02 ± 0.14 | 2.80 ± 0.13 | 2.81 ± 0.17 | 2.95 ± 0.19 |
| 24 hr | | | | |
| Drip loss | 5.95 ± 0.17 | 5.91 ± 0.12 | 5.80 ± 0.17 | 5.92 ± 0.30 |
| 48 hr | | | | |
| Cooking | 43.62 ± 0.72 | 37.56 ± 0.37* | 41.94 ± 0.53** | 41.28 ± |
| loss % | | | | 0.70** |

*significant difference, **extreme difference.

Meat quality analysis: Formula I group compared to control group, the degree of red (A*) increased significantly (P<0.05); Formula I group compared to control group, pH₂₄ was significantly increased (P<0.05); Formula I group compared to control group, lower shear force, which has significantly improved tenderness (P<0.05). L-MA and H-MA group cooking loss significantly decreased 13.89% and 5.36% respectively (P<0.05).

In conclusion, Formula I (e.g., MA) improved pig growth performance. Grower-pig weight and average daily weight gain significantly increased, and feed conversion efficiency rate was improved as well. Formula I effectivity regulated healthy growth of grower-pig and provided the superior meat quality. Supplementing standard grower-pig feed with about 100 mg to about 200 mg, or about 300 mg (e.g., about 100 to about 200, about 200 to about 300, or about 100 to about 300 mg) of Formula I (e.g., MA) improved grower-pig meat tenderness, reduced meat mass cooking loss, improved the average daily weight gain, feed conversion efficiency rate, and improved meat quality. In other words, administering about 300 mg, about 600 mg, about 900 mg, about 300 mg to about 600 mg, about 600 mg to about 900 mg, or about 300 mg to about 900 mg of Formula I (e.g., MA) to a grower-pig improved grower-pig meat tenderness, reduced meat mass cooking loss, improved the average daily weight gain, feed conversion efficiency rate, and improved meat quality.

### Example 5

### Effect of Formula I on Poultry Productivity, Biochemical Profile and Immune System

The aim of the study was to use Formula I as an antibiotic replacement while tracking poultry productivity and biochemical profile and immune system function.

Experimental preparation: 300 female yellow hair broilers from authorized institute were obtained and divided into three groups (each group had 100 heads of day one hatched chicken with an average weight of 42.1 g).

All groups had standard feed of 1-28 day, and 28-42 day standard feed—no additives or antibiotics. All groups fed five times a day from 7 am to 11 pm.

Conditions: the room temperature was maintained at 33-35° C. the first week, then reduced 0.5° C. each day until 20° C. was achieved, at which point a room temperature of 20° C. was maintained to final; 24 hr lights on in the room; body space was 30 heads/m³ for days 1-14; body space was 15 heads/m³ for days 15-28; and body space was 10 heads/m³ from days 29-end.

Group Configuration:

CON—control group had free cooled boiled drinking water;

MA-12—had 1200 ppm Formula I as drinking water; and

MA-20—had 2000 ppm Formula I as drinking water.

Methods to make 1200 ppm and 2000 ppm solution of Formula I: 4% of TPGS-1000 (d-tocopherol polyethylene glycol 1000 succinate) were well dispersed at 90° C. as basic solution in water, then Formula I was diluted into 4% TPGS-1000 solution to make up a 2% solution, then the 2% solution of Formula I was diluted into cooled boiled water to make up 1200 ppm and 2000 ppm solution for the MS-12 group and the MS-20 group as drinking water.

Jugular vein blood was collected (2-3 mL/head; anticoagulant) at days 15, 30 and 45 from 10 random chickens from each group for biochemical profile testing. Jugular vein blood was collected (3.5 mL/head; anticoagulant) at days 14, 28, and 42 from 10 random chickens from each group for immune testing. All testing used standard ELISA kits.

TABLE 5a
effect of Formula I on chicken weight (g) up to 42 days.

| Group | Day 1 | Day 7 | Day 14 | Day 21 | Day 28 | Day 35 | Day 42 |
|---|---|---|---|---|---|---|---|
| CON | 42.1 | 118.6 | 268.2 | 448.9 | 662.5 | 922.8 | 1225.5 |
| MS-12 | 42.1 | 115.5 | 253.3* | 473.2* | 704.2* | 989.7* | 1307.0* |
| MS-20 | 42.1 | 117 | 251* | 446.7 | 694.8* | 968.1* | 1313.8* |

*significant difference.

Table 5a shows that both MS-12 and MS-20 groups had weight gain that was significantly higher than the control group. Between days 14-35, the MS-12 group had a higher weight gain than the MS-20 group, and during the final week the MS-20 group had a higher weight gain than the MS-12 group. These results suggest that further optimization may be beneficial, but overall, administration of Formula I improved weight gain in poultry as compared to the control group.

TABLE 5b
effect of Formula I on chicken weekly weight gain (g).

| Group | Week 1 | Week 2 | Week 3 | Week 4 | Week 5 | Week 6 |
|---|---|---|---|---|---|---|
| CON | 76.5 | 119.6 | 210.7* | 213.6 | 260.3 | 302.7 |
| MS-12 | 73.4 | 137.8* | 219.7* | 231.1* | 285.5* | 317.25* |
| MS-20 | 79.4 | 134* | 195.65 | 248.2** | 273.28* | 345.75* |

*significance difference,
**extreme difference

Table 5b shows that from the second week the MS-12 group weight gain was significantly higher than the control group. In the MS-20 group, weight gain was higher than the control group from weeks 4, 5, and 6. Comparing the MS-12 and the MS-20 groups, the MS-12 group gained more weight than the MS-20 group in the first few weeks, whereas the MS-20 group gained more weight than the MS-12 group in the later weeks. These results suggest that further optimization may be beneficial, but overall, administration of Formula I improved weight gain in poultry as compared to the control group.

TABLE 5c
effect of Formula I on chicken feed conversion rate (%).
Week Feed conversion rate (%)

| | Week | Week | Week | Week | Week | Week | Weeks |
|---|---|---|---|---|---|---|---|
| Group | 1 | 2 | 3 | 4 | 5 | 6 | 1-6 |
| CON | 1.280 | 1.957 | 1.875 | 2.200 | 2.221 | 2.544 | 2.150 |
| MS-12 | 1.358 | 1.785 | 1.776 | 2.177 | 2.060* | 2.519* | 2.076* |
| MS-20 | 1.275 | 1.896 | 1.891 | 1.894* | 2.064* | 2.302** | 2.005** |

*significance difference,
**extreme difference.

Comparing MS-12, MS-20, and CON groups from Table 5c, it can be seen that incorporating Formula I into poultry feed imparts a superior conversion rate. As can be seen, Formula I can save up to 6.7% feed.

TABLE 5d
effect of Formula I on chicken T lymphocyte transformation rate (SI).
Days (SI)

| Group | 15 days | 30 days | 45 days |
|---|---|---|---|
| CON | 1 | 1 | 1 |
| MS-12 | 1.005 | 4.05** | 1.48* |
| MS-20 | 1.014 | 4.23** | 1.48* |

*significant difference, **extreme difference.

Formula I was evaluated for its effect on the chicken immune system response. Table 5d shows an extremely high T lymphocyte transformation rate for groups administered Formula I as compared to the control group. This was a similar finding to the pig experimental tests which showed that Formula I significantly enhanced and regulated the immune function.

TABLE 5e
influence of Formula I on chicken serum total protein.

| Group | 14 days | 28 days | 42 days |
|---|---|---|---|
| CON | 22.32 | 27.9 | 23.4 |
| MS-12 | 27.40* | 30* | 27.4* |
| MS-20 | 25.0** | 28.9 | 25.0 |

*significant difference, **extreme difference.

TABLE 5f
influence of Formula I on chicken serum total globulin.

| Group | 14 days | 28 days | 42 days |
|---|---|---|---|
| CON | 2.09 | 6.9 | 3.71 |
| MS-12 | 6** | 7.93* | 6.82** |
| MS-20 | 5.45** | 6.62 | 5.41** |

*significant difference, **extreme difference.

TABLE 5g
influence of Formula I on chicken serum IgG (ng/mL).

| Group | 14 days | 28 days | 42 days |
|---|---|---|---|
| CON | 105.8 | 82.7 | 84.2 |
| MS-12 | 122.1* | 77.5 | 81.6 |
| MS-20 | 137.2* | 82.5 | 80.6 |

*significant difference.

TABLE 5h
influence of Formula I on chicken total antioxidant capacity (u/mL).

| Group | 14 days | 28 days | 42 days |
|---|---|---|---|
| CON | 17.7 | 16.7 | 16.9 |
| MS-12 | 22.8* | 15.9 | 21.4* |
| MS-20 | 22.9* | 17.5 | 17.6 |

*significant difference.

From Tables 5e-5h of chicken biochemical profiles during administration of Formula I, the MS group serum total globulin levels over days 14-42 are significantly superior to that of the control group, and IgG levels are supportive of this. This data suggests that Formula I significantly enhanced the chicken humoral immune function and immune system regulation.

Antibodies have had an enormous impact as a basic research tool and are increasingly proving themselves to have remarkable efficacy. In large scale chicken farm operations, Newcastle Disease Virus (NDV), avian Infectious Bronchitis Virus (IBV), and Infectious Bursal Disease Virus (IBDV) antibodies are often used in chicken farm protecting procedures. However, the above data shows that administration of compositions provided herein (e.g., compositions comprising Formula I) may be a suitable supplement or alternative to standard livestock antibody therapy.

### Example 6

### Antiviral Effect of Formula I Combined with Antibody Therapy

The aim of the study was to examine the influence of administering Formula I to poultry in a poultry production farm that otherwise regularly uses vaccination with NDV antibody, IBV antibody, and IBDV antibody to innoculate the flocks. Titer values for NDV antibodies, IBV antibodies, and IBDV antibodies were assessed following administration of Formula I.

Experimental preparation: 300 female yellow hair broilers from authorized institute were obtained and divided into three groups (each group had 100 heads of day one hatched chicken with an average weight of 42.1 g); fed 45 days.

All groups had standard feed of 1-28 day, and 28-42 day standard feed—no additives or antibiotics. All groups fed five times a day from 7 am to 11 pm.

Conditions: the room temperature was maintained at 33-35° C. the first week, then reduced 0.5° C. each day until 20° C. was achieved, at which point a room temperature of 20° C. was maintained to final; 24 hr lights on in the room; body space was 30 heads/m³ for days 1-14; body space was 15 heads/m³ for days 15-28; and body space was 10 heads/m³ from days 29-end.

Group Configuration:

CON—control group had free cooled boiled drinking water;

MA-12—had 1200 ppm Formula I as drinking water; and

MA-20—had 2000 ppm Formula I as drinking water.

Methods to make 1200 ppm and 2000 ppm solution of Formula I: 4% of TPGS-1000 (d-tocopherol polyethylene glycol 1000 succinate) were well dispersed at 90° C. as basic solution in water, then Formula I was diluted into 4% TPGS-1000 solution to make up a 2% solution, then the 2% solution of Formula I was diluted into cooled boiled water to make up 1200 ppm and 2000 ppm solution for the MS-12 group and the MS-20 group as drinking water.

On the third day eye-drops with NDV antibody and IBV antibody were administered. On day 14 IBDV antibody mixed drinking water was administered, and on day 24 NDV antibody mixed drinking water was administered.

Jugular vein blood was collected (3.5 mL/head; non-anticoagulative) at days 14, 28, and 42 from 10 random chickens from each group for hemagglutination inhibition (HA/HI) to determine antibody titers. Likewise, serum was collected on days 28 and 42 to determine IBDV antibody titer. The influence of administration of Formula I on NDV antibody titer, IBV antibody titer, and IBDV antibody titer was assessed. The results are summarized in Tables 6a-6c.

TABLE 6a
influence of Formula I on Newcastle Disease Virus antibody titer (log2).

| Group | 14 days | 28 days | 42 days |
|---|---|---|---|
| CON | 5.6 | 5.7 | 5.8 |
| MS-12 | 6.4* | 7.1* | 6.2* |
| MS-20 | 6* | 8.3* | 8.3* |

*significant difference.

TABLE 6b
influence of Formula I on Avian Infectious
Bronchitis Virus antibody titer (log2).

| Group | 14 days | 28 days | 42 days |
|---|---|---|---|
| CON | 5.2 | 5.7 | 5.7 |
| MS-12 | 6.2* | 6.8* | 6.7* |
| MS-20 | 6.4* | 6.5* | 6.5* |

*significant difference.

TABLE 6c
influence of Formula I on Infectious Bursal
Disease Virus antibody titer (log2).

| Group | 28 days | 42 days |
|---|---|---|
| CON | 5.4 | 5.3 |
| MS-12 | 6.8* | 6.5* |
| MS-20 | 6.6* | 6.5* |

*significant difference.

The results of Tables 6a-6c indicate that Formula I, when combined with antibody therapy, significantly improved the NDV antibody titer, IBV antibody titer, and IBDV antibody titer. Thus, in some embodiments, a composition comprising about 1000 ppm to about 2500 ppm, about 1200 ppm to about 2000 ppm, about 1000 ppm , about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, or a range bounded by any two of these values of Formula I is administered to a subject. In some embodiments the subject is in need of treatment of a viral infection. In some embodiments, the composition is an aqueous composition. In some embodiments, the aqueous composition is suitable for drinking water for the subject.

### Example 7

### Gas Chromatography Analysis of Formula I

Formula I, as prepared in Example 1 vide supra, is analyzed by gas chromatography according to one of the following three parameter sets in accordance with ISO 4730:2004:
1. Column: fused silica capillary, length 50 m, internal diameter 0.20 mm;
   Oven temperature: temperature programming from 70° C. to 220° C. at 2° C./min;
   Injector temperature: 230° C.;
   Detector temperature: 250° C.;
   Detector: flame ionization type;
   Carrier gas: helium;
   Volume injected: 0.2 μL; and
   Carrier gas flow rate: 1.0 mL/min, Split ratio: 1/100.
2. Column: FSOT (fused silica open tubular), length 60 m, internal diameter 0.25 mm;
   Oven temperature: temperature programming from 50° C. to 250° C. at 10° C./min, and isothermal at 250° C. for 9 min;
   Injector temperature: 200° C.;
   Detector temperature: 300° C.;
   Detector: flame ionization type;
   Carrier gas: helium;
   Volume injected: 1.0 μL; and
   Carrier gas flow rate: 1.0 mL/min, Split ratio: 1/50.
3. Column: fused silica capillary, length 50 m, internal diameter 0.33 mm;
   Oven temperature: isothermal at 50° C. for 1 minute, then temperature programming from 50° C. to 220° C. at 5° C./min, and isothermal at 220° C. for 5 min;
   Injector temperature: 240° C.;
   Detector temperature: 240° C.;
   Detector: flame ionization type;
   Carrier gas: helium;
   Volume injected: 1.5 μL; and
   Carrier gas flow rate: 1.0 mL/min, Split ratio: 1/100.

### INCORPORATION BY REFERENCE AND EQUIVALENTS

The contents of all references (including literature references, issued patents, published patent applications, and co-pending patent applications) cited throughout this application are hereby expressly incorporated herein by reference in their entireties. Unless otherwise defined, all technical and scientific terms used herein are accorded the meaning commonly known to one of ordinary skill in the art.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Those skilled in the art will recognize, or be able to ascertain using no more than routine experimentation, many equivalents of the specific embodiments provided herein. Such equivalents are intended to be encompassed by the following claims.

What is claimed is:

1. A composition comprising:
α-terpinolene;
terpinen-4-ol; and
α-terpineol;
wherein the molar ratio of α-terpinolene:terpinen-4-ol:α-terpineol is about 1:(about 2.15 to about 3.65):(about 0.22 to about 0.65).

2. The composition of claim 1, wherein the molar ratio of α-terpinolene:terpinen-4-ol:α-terpineol is about 1:(about 2.35 to about 3.25):(about 0.27 to about 0.52).

3. The composition of claim 1 or claim 2, wherein the molar ratio of α-terpinolene:terpinen-4-ol:α-terpineol is about 1:(about 2.5 to about 2.85):(about 0.32 to about 0.45).

4. The composition of any one of claims 1 to 3, wherein the composition comprises about 17 to about 25 A_{GC}% α-terpinolene.

5. The composition of any one of claims 1 to 4, wherein the composition comprises about 19.6 A_{GC}% α-terpinolene.

6. The composition of any one of claims 1 to 5, wherein the composition comprises about 45 to about 58 A_{GC}% terpinen-4-ol.

7. The composition of any one of claims 1 to 6, wherein the composition comprises about 52.5 A_{GC}% terpinen-4-ol.

8. The composition of any one of claims 1 to 7, wherein the composition comprises about 2.0 to about 11 A_{GC}% α-terpineol.

9. The composition of any one of claims 1 to 8, wherein the composition comprises about 9.2 A_{GC}% α-terpineol.

10. The composition of any one of claims 1 to 9, wherein the composition comprises about 0.5 to about 2.5 A_{GC}% 1,8-cineole.

11. The composition of any one of claims 1 to 10, wherein the composition comprises about 1.8 A_{GC}% 1,8-cineole.

12. The composition of any one of claims 1 to 11, wherein the composition comprises about 0.5 to about 2.5 A_{GC}% γ-terpinene.

13. The composition of any one of claims 1 to 12, wherein the composition comprises about 2.2 A_{GC}% γ-terpinene.

14. The composition of any one of claims 1 to 13, wherein the composition comprises α-pinene up to about 1.7 A_{GC}%.

15. The composition of any one of claims 1 to 14, wherein the composition comprises about 1.6 A_{GC}% α-pinene.

16. The composition of any one of claims 1 to 15, wherein the composition comprises sabinene up to about 0.2 A_{GC}%.

17. The composition of any one of claims 1 to 16, wherein the composition comprises sabinene up to about 0.01 A_{GC}%.

18. The composition of any one of claims 1 to 17, wherein the composition comprises α-terpinene up to about 0.2 A_{GC}%.

19. The composition of any one of claims 1 to 18, wherein the composition comprises α-terpinene up to about 0.01 A_{GC}%.

20. The composition of any one of claims 1 to 19, wherein the composition comprises limonene up to about 0.2 A_{GC}%.

21. The composition of any one of claims 1 to 20, wherein the composition comprises limonene up to about 0.01 A_{GC}%.

22. The composition of any one of claims 1 to 21, wherein the composition comprises p-cymene up to about 0.2 A_{GC}%.

23. The composition of any one of claims 1 to 22, wherein the composition comprises p-cymene up to about 0.01 A_{GC}%.

24. The composition of any one of claims 1 to 23, wherein the composition comprises aromadendren up to about 0.5 A_{GC}%.

25. The composition of any one of claims 1 to 24, wherein the composition comprises aromadendren up to about 0.01 A_{GC}%.

26. The composition of any one of claims 1 to 25, wherein the composition comprises ledene up to about 0.5 A_{GC}%.

27. The composition of any one of claims 1 to 26, wherein the composition comprises ledene up to about 0.01 A_{GC}%.

28. The composition of any one of claims 1 to 27, wherein the composition comprises δ-cadinene up to about 0.5 A_{GC}%.

29. The composition of any one of claims 1 to 28, wherein the composition comprises δ-cadinene up to about 0.01 A_{GC}%.

30. The composition of any one of claims 1 to 29, wherein the composition comprises globulol up to about 0.5 A_{GC}%.

31. The composition of any one of claims 1 to 30, wherein the composition comprises globulol up to about 0.01 A_{GC}%.

32. The composition of any one of claims 1 to 31, wherein the composition comprises virdiflorol up to about 0.5 A_{GC}%.

33. The composition of any one of claims 1 to 32, wherein the composition comprises virdiflorol up to about 0.01 A_{GC}%.

34. The composition of any one of claims 1 to 33, wherein the composition comprises about 17 to about 25 A_{GC}% α-terpinolene, about 45 to about 58 A_{GC}% terpinen-4-ol, about 2.0 to about 11 A_{GC}% α-terpineol, about 0.5 to about 2.5 A_{GC}% 1,8-cineole, or about 0.5 to about 2.5 A_{GC}% γ-terpinene.

35. The composition of any one of claims 1 to 34, wherein the composition comprises about 17 to about 25 A_{GC}% α-terpinolene, about 45 to about 58 A_{GC}% terpinen-4-ol, and about 2.0 to about 11 A_{GC}% α-terpineol.

36. The composition of any one of claims 1 to 35, wherein the composition comprises about 17 to about 25 A_{GC}% α-terpinolene, about 45 to about 58 A_{GC}% terpinen-4-ol, about 2.0 to about 11 A_{GC}% α-terpineol, about 0.5 to about 2.5 A_{GC}% 1,8-cineole, and about 0.5 to about 2.5 A_{GC}% γ-terpinene.

37. The composition of any one of claims 1 to 36, wherein the composition further comprises at least one of:
α-pinene up to about 1.7 A_{GC}%;
sabinene up to about 0.2 A_{GC}%;
α-terpinene up to about 0.2 A_{GC}%;
limonene up to about 0.2 A_{GC}%;
p-cymene up to about 0.2 A_{GC}%;
aromadendrene up to about 0.5 A_{GC}%;
ledene up to about 0.5 A_{GC}%;
δ-cadinene up to about 0.5 A_{GC}%;
globulol up to about 0.5 A_{GC}%; or
virdiflorol up to about 0.5 A_{GC}%.

38. The composition of any one of claims 1 to 37, wherein the composition comprises:
about 17 to about 25 A_{GC}% α-terpinolene;
about 45 to about 58 A_{GC}% terpinen-4-ol;
about 2.0 to about 11 A_{GC}% α-terpineol;
about 0.5 to about 2.5 A_{GC}% 1,8-cineole;
about 0.5 to about 2.5 A_{GC}% γ-terpinene;
α-pinene up to about 1.7 A_{GC}%;
sabinene up to about 0.2 A_{GC}%;
α-terpinene up to about 0.2 A_{GC}%;
limonene up to about 0.2 A_{GC}%;
p-cymene up to about 0.2 A_{GC}%;
aromadendrene up to about 0.5 A_{GC}%;
ledene up to about 0.5 A_{GC}%;
δ-cadinene up to about 0.5 A_{GC}%;
globulol up to about 0.5 A_{GC}%; and
virdiflorol up to about 0.5 A_{GC}%.

39. The composition of any one of claims 1 to 38, wherein the α-terpinolene and terpinen-4-ol together are at least about 62 A_{GC}% of the composition.

40. The composition of any one of claims 1 to 39, wherein the α-terpinolene and terpinen-4-ol together are at least about 72 A_{GC}% of the composition.

41. The composition of any one of claims 1 to 40, wherein the α-terpinolene and terpinen-4-ol together are about 72 to about 83 A_{GC}% of the composition.

42. The composition of any one of claims 1 to 39, wherein the α-terpinolene and terpinen-4-ol together are about 62 to about 83 A_{GC}% of the composition.

43. The composition of any one of claims 1 to 39, wherein the α-terpinolene and terpinen-4-ol together are about 70 to about 75 A_{GC}% of the composition.

44. The composition of any one of claims 1 to 43, wherein the α-terpinolene, terpinen-4-ol, and α-terpineol together are at least about 62.5 A_{GC}% of the composition.

45. The composition of any one of claims 1 to 44, wherein the α-terpinolene, terpinen-4-ol, and α-terpineol together are at least about 78 A_{GC}% of the composition.

46. The composition of any one of claims 1 to 45, wherein the α-terpinolene, terpinen-4-ol, and α-terpineol together are about 78 to about 85.5 A_{GC}% of the composition.

47. The composition of any one of claims 1 to 44, wherein the α-terpinolene, terpinen-4-ol, and α-terpineol together are about 62.5 to about 85.5 A_{GC}% of the composition.

48. The composition of any one of claims 1 to 44, wherein the α-terpinolene, terpinen-4-ol, and α-terpineol together are about 75.5 to about 81 A_{GC}% of the composition.

49. The composition of any one of claims 1 to 48, wherein the composition comprises:
about 19.6 A_{GC}% α-terpinolene;
about 52.5 A_{GC}% terpinen-4-ol;
about 9.2 A_{GC}% α-terpineol;
about 1.8 A_{GC}% 1,8-cineole;
about 2.2 A_{GC}% γ-terpinene; and
α-pinene about 1.6 A_{GC}%.

50. The composition of any one of claims 1 to 48, wherein the composition comprises:
about 19.6 A_{GC}% α-terpinolene;
about 52.5 A_{GC}% terpinen-4-ol;
about 9.2 A_{GC}% α-terpineol;
about 1.8 A_{GC}% 1,8-cineole;
about 2.2 A_{GC}% γ-terpinene;
α-pinene about 1.6 A_{GC}%;
sabinene up to about 0.01 A_{GC}%;
α-terpinene up to about 0.01 A_{GC}%;
limonene up to about 0.01 A_{GC}%;
p-cymene up to about 0.01 A_{GC}%;
aromadendrene up to about 0.01 A_{GC}%;
ledene up to about 0.01 A_{GC}%;
δ-cadinene up to about 0.01 A_{GC}%;
globulol up to about 0.01 A_{GC}%; and
virdiflorol up to about 0.01 A_{GC}%.

51. The composition of any one of claims 1 to 50, wherein the composition is a pharmaceutical composition.

52. The composition of any one of claims 1 to 51, wherein the composition further comprises at least one pharmaceutically acceptable excipient.

53. A method of decreasing the rate of mortality in a subject in need thereof, comprising administering the composition of any one of claims 1 to 52 to the subject.

54. A method of decreasing the rate of bacterial infection in a subject in need thereof, comprising administering the composition of any one of claims 1 to 52 to the subject.

55. A method of decreasing the rate of diarrhea in a subject in need thereof, comprising administering the composition of any one of claims 1 to 52 to the subject.

56. A method of decreasing the rate of premature death in a subject in need thereof, comprising administering the composition of any one of claims 1 to 52 to the subject.

57. The method of any one of claims 53 to 56, wherein the subject is a clade.

58. The method of any one of claims 53 to 57, wherein the subject is a clade of livestock.

59. The method of any one of claims 53 to 58, wherein the subject is a livestock.

60. The method of any one of claims 53 to 59, wherein the subject is a human being.

61. A method of treating diarrhea in a subject in need thereof, comprising administering the composition of any one of claims 1 to 52 to the subject.

63. The method of claim 61 or claim 62, wherein the subject is a livestock.

64. The method of claim 61 or claim 62, wherein the subject is a human being.

65. The method of any one of claims 53 to 64, wherein the composition is administered systemically.

66. The method of any one of claims 53 to 65, wherein the composition is administered by enteral administration.

67. The method of any one of claims 53 to 66, wherein the composition is administered by oral administration, gastric feeding tube, duodenal feeding tube, gastrostomy, rectal administration, or vaginal administration.

68. The method of any one of claims 53 to 67, wherein the composition is administered by oral administration.

69. The method of any one of claims 53 to 65, wherein the composition is administered by parenteral administration.

70. The method of any one of claims 53 to 65 and 68, wherein the composition is administered by intramuscular administration, subcutaneous administration, intravenous administration, intradermal administration, intra-arterial administration, or intraosseous administration.

71. The method of any one of claims 53 to 64, wherein the composition is administered topically.

72. The method of any one of claims 53 to 64 and 71, wherein the composition is administered by epicutaneous administration, inhalation, enema, ophthalmic administration, otic administration, or nasal administration.
